(19) United States
(12) Patent Application Publication
(10) Pub. No.: US 2026/0285868 A1
(43) Pub. Date: Sep. 24, 2026

(54) IMIDAZOTRIAZINE DERIVATIVES AS IL-17 MODULATORS

(71) Applicant: UCB BIOPHARMA SRL, Brussels (BE)

(72) Inventors: Gregory William HASLETT, Slough Berkshire (GB); James Thomas REUBERSON, Slough Berkshire (GB)

(21) Appl. No.: 19/475,113

(22) PCT Filed: Apr. 15, 2024
(86) PCT No.: PCT/EP2024/060131

§ 371 Date: Oct. 14, 2025

(30) Foreign Application Priority Data
Apr. 17, 2023 (GB) 2305585.8

(51) Int. Cl. C07D 487/04; A61K 31/53; C07B 59/00

(52) U.S. Cl. CPC C07D 487/04; A61K 31/53; C07B 59/002

(57) ABSTRACT

A compound of formula (I) as defined herein, or a pharmaceutically acceptable salt thereof, being potent modulators of human IL-17 activity, are accordingly of benefit in the treatment and/or prevention of various human ailments, including inflammatory and autoimmune disorders.

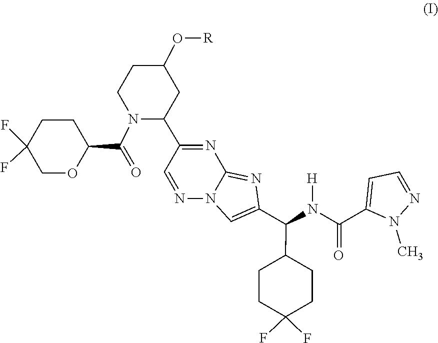

The present invention relates to heterocyclic compounds, and to their use in therapy. More particularly, this invention is concerned with pharmacologically active substituted imidazo[1,2-b][1,2,4]triazine derivatives. These compounds act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J. Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Korn et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2023/275301, and also WO 2020/261141, describe fused bicyclic imidazole derivatives that are stated to act as modulators of IL-17 activity, and thus to be of benefit in the treatment of pathological conditions including adverse inflammatory and autoimmune disorders.

None of the prior art available to date, however, discloses or suggests the precise structural class of substituted imidazo[1,2-b][1,2,4]triazine derivatives as provided by the present invention.

As well as being potent modulators of human IL-17 activity, the compounds in accordance with the present invention possess other notable advantages. In particular, the compounds of the invention display valuable metabolic stability, as determined in either microsomal or hepatocyte incubations. The compounds of the invention also display valuable permeability as determined by standard assays, e.g. the Caco-2 permeability assay.

The present invention provides a compound of formula (I), or a pharmaceutically acceptable salt thereof:

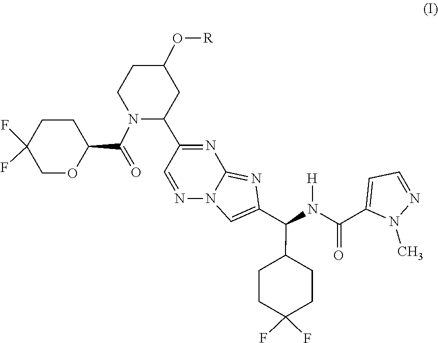

wherein R represents methyl.

The compounds in accordance with the present invention are encompassed within the generic scope of WO 2023/275301. There is, however, no specific disclosure therein of a compound of formula (I) as defined above, or a pharmaceutically acceptable salt thereof.

The present invention also provides a compound of formula (I) as defined above, or a pharmaceutically acceptable salt thereof, for use in therapy.

The present invention also provides a compound of formula (I) as defined above, or a pharmaceutically acceptable salt thereof, for use in the treatment and/or prevention of disorders for which the administration of a modulator of IL-17 function is indicated.

The present invention also provides the use of a compound of formula (I) as defined above, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above, or a pharmaceutically acceptable salt thereof.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P.H. Stahl & C.G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

Formula (I) and the formulae depicted hereinafter are intended to represent all individual stereoisomers and all possible mixtures thereof, unless stated or shown otherwise. In addition, compounds of formula (I) may exist as tautomers, for example amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium, D) or ³H (tritium, T) atom, preferably ¹H. Similarly, by way of example, each individual carbon atom present in formula (I), or in the formulae depicted hereinafter, may be present as a ¹²C, ¹³C or ¹⁴C atom, preferably ¹²C.

Suitably, R represents CH₃ or CD₃.

In a first embodiment, R represents CH₃. In a second embodiment, R represents CD₃.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, axial spondyloarthritis, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, hidradenitis suppurativa, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydia and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, hidradenitis suppurativa, scleroderma, systemic sclerosis, lung fibrosis, inflammatory bowel diseases (including Crohn's disease and ulcerative colitis), axial spondyloarthritis, ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis, hidradenitis suppurativa, axial spondylo-arthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above may be prepared by a process which comprises reacting (2S)-5,5-difluorotetrahydropyran-2-carboxylic acid with the compound of formula (II):

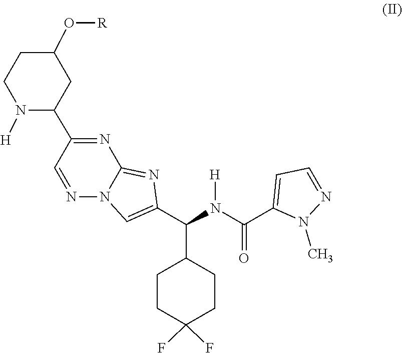

wherein R is as defined above.

The reaction is conveniently accomplished in the presence of a coupling agent and a base. Suitable coupling agents include 1-[bis(dimethylamino)methylene]-1H-1,2,3-triazolo[4,5-b]pyridinium 3-oxid hexafluorophosphate (HATU); and 2,4,6-tripropyl-1,3,5,2,4,6-trioxatriphosphorinane-2,4,6-trioxide; and 2-chloro-1-methylpyridinium iodide. Suitable bases include organic amines, e.g. a trialkylamine such as N,N-diisopropylethylamine; or pyridine. The reaction is conveniently performed at ambient or elevated temperature in a suitable solvent, e.g. a cyclic ether such as tetrahydrofuran; or a dipolar aprotic solvent such as N,N-dimethylformamide or N,N-dimethylacetamide; or a chlorinated solvent such as dichloromethane; or an organic ester solvent such as ethyl acetate.

The intermediate of formula (II) above may be prepared by removal of the N-protecting group R^{q} from a compound of formula (III):

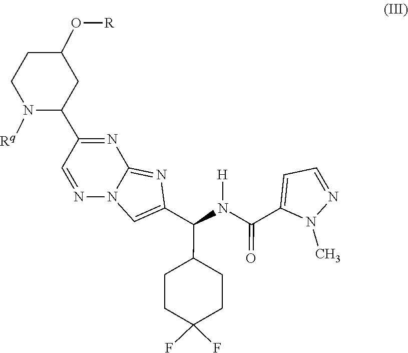

wherein R is as defined above, and R^{q} represents a N-protecting group.

The N-protecting group R^{q} will suitably be tert-butoxycarbonyl (BOC), in which case the removal thereof may conveniently be effected by treatment with an acid, e.g. a mineral acid such as hydrochloric acid, or an organic acid such as trifluoroacetic acid.

The intermediates of formula (III) above may be prepared by reacting a compound of formula (IV) with the compound of formula (V):

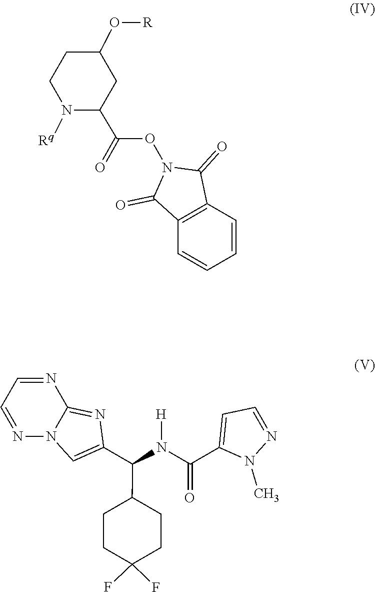

wherein R and R^{q} are as defined above; in the presence of a transition metal catalyst.

Suitable transition metal catalysts of use in the reaction include [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate; and tris[2-phenylpyridinato-C²,N]iridium(III). The reaction will generally be performed by exposing the reactants to a bright light source. A suitable bright light source will typically comprise the ‘integrated photoreactor’ described in ACS Cent. Sci., 2017, 3, 647-653; or the Penn Photoreactor M2 or M1 system. The reaction will conveniently be carried out at ambient temperature in the presence of trifluoroacetic acid and in a suitable solvent, e.g. a dipolar aprotic solvent such as N,N-dimethylformamide, or an organic sulfoxide such as dimethyl sulfoxide.

The intermediate of formula (V) above may be prepared by a process which comprises reacting 1-methyl-1H-pyrazole-5-carboxylic acid with the compound of formula (VI):

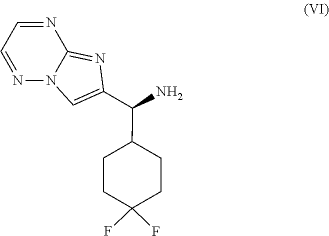 [SMILES: N[C@H](c1cn2nccnc2n1)C1CCC(F)(F)CC1]

under conditions analogous to those described above for the reaction between compound (II) and (2S)-5,5-difluorotetrahydropyran-2-carboxylic acid.

The intermediate of formula (VI) above may be prepared by the procedure described in WO 2023/275301, or by methods analogous thereto.

The intermediates of formula (IV) above may be prepared by reacting a carboxylic acid derivative of formula (VII):

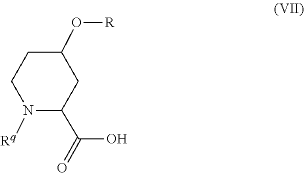

wherein R is as defined above; with N-hydroxyphthalimide.

The reaction may conveniently be accomplished in the presence of a coupling agent such as N-(3-dimethylaminopropyl)-N′-ethylcarbodiimide hydrochloride. The reaction is suitably performed at ambient temperature, optionally in the presence of 4-(dimethylamino)pyridine, and in a suitable solvent, e.g. a chlorinated solvent such as dichloromethane.

Where they are not commercially available, the starting materials of formula (VII) may be prepared by methods analogous to those described in the accompanying Examples, or by standard methods well known from the art.

Where a mixture of products is obtained from any of the processes described above for the preparation of a compound according to the invention, the desired product can be separated therefrom at an appropriate stage by conventional methods such as preparative HPLC; or column chromatography utilising, for example, silica and/or alumina in conjunction with an appropriate solvent system.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit IL-17 induced IL-6 release from human dermal fibroblasts. Thus, when tested in the HDF cell line assay described below, the compounds of the present invention generally exhibit a pIC₅₀ value of 6.5 or more, usually of 7.0 or more, typically of 7.2 or more, suitably of 7.5 or more, ideally of 7.8 or more, and preferably of 8.0 or more (pIC₅₀ equals −log₁₀[IC₅₀], in which IC₅₀ is expressed as a molar concentration, so the skilled person will appreciate that a higher pIC₅₀ figure denotes a more active compound).

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 μL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

10 μL from the aqueous dilution plate was transferred to the reagent plate containing 30 μL of the diluted cytokines, to give a 2.5% DMSO solution. The compounds were incubated with the cytokine mixtures for 5 h at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 L), then supernatant (10 L) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm).

When tested in the HDF cell line assay as described above, the compounds of the accompanying Examples were found to exhibit the following pIC₅₀ values.

| Example | pIC₅₀ |
|---|---|
| 1 (Peak 1) | 7.4 |
| 2 (Peak 2) | 8.4 |
| 3 (Peak 1) | 7.4 |
| 4 (Peak 2) | 8.5 |

### Comparative Data

By way of comparison, the title compounds of Examples 46 and 47 of WO 2023/275301 are stated therein to exhibit pIC₅₀ values of 7.9 and 6.2 respectively.

The following Examples illustrate the preparation of compounds according to the invention.

### EXAMPLES

### Abbreviations

DCM: dichloromethane DMSO: dimethyl sulfoxide
DIPEA: N,N-diisopropylethylamine DMF: N,N-dimethylformamide
EtOAc: ethyl acetate TFA: triflouroacetic acid
MeOH: methanol THF: tetrahydrofuran
EDCI.HCl: N-(3-dimethylaminopropyl)-N′-ethylcarbodiimide hydrochloride
HATU: 1-[bis(dimethylamino)methylene]-1H-1,2,3-traizolo[4,5-b]pyridinium 3-oxid hexafluorophosphate
{Ir[dF(CF₃)ppy]₂(dtbpy)}PF₆: [4,4′-bis(1,1-dimethylethyl)-2,2′-bipydridine-N1,N1′]bis-{3,5-difluoro-2-[5-(trifluoromethyl)-2-pyridinyl-N]phenyl-C}iridium(III) hexafluoro-phosphate
h: hour r.t.: room temperature
M: mass; molar RT: retention time
HPLC: High Performance Liquid Chromatography
LCMS: Liquid Chromatography Mass Spectrometry
SFC: Supercritical Fluid Chromatography
LED: light-emitting diode
TOF-MS: Time-of-Flight Mass Spectrometry

### Analytical and Separation Methods

### Method 1

Stationary Phase: Phenomenex Gemini NX-C18 2×20 mm, 3 μm
Column Temperature: 40° C.
Mobile Phase A: 10 mM ammonium formate in water+0.1% ammonia solution
Mobile Phase B: acetonitrile+5% water+0.1% ammonia solution
Flow rate: 1 mL/minute
Gradient program: Time A % B %
   0.00 95.00 5.00
   1.50 5.00 95.00
   2.25 5.00 95.00
   2.50 95.00 5.00

### Method 2

Stationary Phase: Phenomenex Gemini NX-C18 2×20 mm, 3 μm
Column Temperature: 40° C.
Mobile Phase A: 10 mM ammonium formate in water+0.1% ammonia solution
Mobile Phase B: acetonitrile+5% water+0.1% ammonia solution
Flow rate: 1 mL/minute
Gradient program: Time A % B %
   0.00 95.00 5.00
   4.00 5.00 95.00
   5.00 5.00 95.00
   5.10 95.00 5.00

### Method 3

Stationary Phase: Waters Acquity UPLC BEH C18 2.1×50 mm, 1.7 μm
Mobile Phase A: 10 mM ammonium formate in water+0.1% ammonia solution
Mobile Phase B: acetonitrile+5% water+0.1% ammonia solution
Flow rate: 1.5 mL/minute
Gradient program: Time A % B %
   0.00 95.00 5.00
   0.10 95.00 5.00
   3.50 5.00 95.00
   4.00 5.00 95.00
   4.05 95.00 5.00

### Method 4

Chiral analysis by SFC (Lux Cellulose-1, 150×4.6 mm, 3 μm column, flow rate 3 mL/minute, 120 bar, column temperature 35° C.) eluting with a 3-40% MeOH (+0.1% NH₄OH) method, using a 6.5 minute run time on a Waters UPC² Acquity system.

### Intermediate 1

### N—[(S)-(4,4-Difluorocyclohexyl)(imidazo[1,2-b][1,2,4]triazin-6-yl)methyl]-2-methyl-pyrazole-3-carboxamide

(S)-(4,4-Difluorocyclohexyl)(imidazo[1,2-b][1,2,4]triazin-6-yl)methanamine (WO 2023/275301, Intermediate 20) (4.5 g, 15 mmol) and 1-methyl-1H-pyrazole-5-carboxylic acid (2.9 g, 23 mmol) were dissolved in DMF (70 mL), then treated with DIPEA (4.0 mL, 23 mmol) and HATU (8.8 g, 23 mmol). The mixture was stirred for 18 h, then separated between ethyl acetate (100 mL) and brine (100 mL). The organic layer was washed with brine (4×40 mL), then passed through a phase separator and evaporated in vacuo. The crude residue was purified by flash column chromatography, eluting with a gradient of 1-100% EtOAc in hexanes, to afford the title compound (4.2 g, 70%) as a yellow/orange solid. LCMS (Method 2): [M+H]⁺ m/z 376.2, RT 1.53 minutes.

### Intermediate 2

### 1-tert-Butoxycarbonyl-4-hydroxypiperidine-2-carboxylic acid

Into a flame-dried 100 mL, three-neck round-bottomed flask under nitrogen were charged (R)-1-tert-butoxycarbonyl-4-oxopiperidine-2-carboxylic acid (2.00 g, 8.22 mmol) and THE (10 mL). The stirred solution was cooled to −72° C. (internal temperature) and lithium tri-sec-butylborohydride (1.0M in TIF) (12 mL, 12 mmol) was added dropwise, maintaining the internal temperature below −60° C. After complete addition, the reaction mixture was warmed to −5° C. and stirred at −10° C. to 0° C. (internal temperature) for 3 h. The cooled reaction mixture was quenched with 1M aqueous sodium hydroxide solution (20 mL), followed by cautious addition of hydrogen peroxide solution (30% in water) (10 mL), maintaining the internal temperature below 25° C. The mixture was stirred at r.t. for 1 h, then ethyl acetate (20 mL) and water (20 mL) were added. The mixture was stirred at r.t. overnight. The aqueous layer was separated and acidified to pH 1-2 with 1M aqueous hydrochloric acid, then extracted with ethyl acetate (2×100 mL). The combined organic extracts were washed with water (2×100 mL) and brine (100 mL), then passed through a phase separator and concentrated in vacuo. The crude material was dissolved in hot ethyl acetate (5 mL), then isohexane (20 mL) was added and the solution was allowed to stand at r.t. for 1 h. To the precipitated solid was added further isohexane (20 mL). The solid was filtered, then washed with isohexane and dried under vacuum, to give the title compound (~9:1 ratio of stereoisomers) (1.40 g, 69%) as a white solid. δ_{H} (400 MHz, CDCl₃) (peaks reported for major stereoisomer only) 6.00-5.17 (m, 2H), 4.90-4.58 (m, 1H), 4.23-4.12 (m, 1H), 3.95-3.69 (m, 1H), 3.48-3.24 (m, 1H), 2.52-2.34 (m, 1H), 1.91 (ddd, J 14.6, 6.9, 2.3 Hz, 1H), 1.83-1.58 (m, 2H), 1.44 (s, 9H).

### Intermediate 3

### 1-tert-Butoxycarbonyl-4-(trideuteriomethoxy)piperidine-2-carboxylic acid

Sodium hydride (60 mass % in mineral oil) (983 mg, 24.6 mmol) was added to a solution of Intermediate 2 (~9:1 ratio of stereoisomers) (2.74 g, 11.18 mmol) in THE (112 mL) under nitrogen at 0° C. The ice bath was removed, and the reaction mixture was stirred at r.t. for 1 h. The reaction mixture was cooled to 0° C. and iodomethane-d3 (1.53 mL, 24.6 mmol) was added dropwise. The reaction mixture was stirred with warming to 5° C. (by allowing the ice in the ice bath to melt) for 3 h, then at r.t. for a further 19 h. The reaction mixture was quenched with water (100 mL) and washed with DCM (70 mL). The DCM layer was discarded. The aqueous layer was acidified to pH 1-2 with 2M aqueous hydrochloric acid (~8 mL) and extracted with DCM (5×70 mL). The organic extracts were combined, then passed through a phase separator and concentrated in vacuo, to give the title compound (~8:1 ratio of stereoisomers) (2.99 g, quantitative) as a yellow oil, which was utilised without further purification. δ_{H} (400 MHz, DMSO-d₆) (peaks reported for major isomer only) 12.44 (br s, 1H), 4.41 (dd, J 31.8, 6.6 Hz, 1H), 3.69-3.57 (m, 1H), 3.50-3.44 (m, 1H), 3.27-2.99 (m, 1H), 2.47-2.31 (m, 1H), 1.82-1.65 (m, 2H), 1.49 (tdd, J 13.6, 5.3, 2.6 Hz, 1H), 1.44-1.30 (m, 9H).

### Intermediate 4

### O¹-tert-Butyl O²-(1,3-dioxoisoindolin-2-yl) 4-(trideuteriomethoxy)piperidine-1,2-dicarboxylate

EDCI·HCl (2.356 g, 12.29 mmol) was added to a solution of Intermediate 3 (~8:1 ratio of stereoisomers) (2.99 g, 11.17 mmol) and N-hydroxyphthalimide (2.06 g, 12.29 mmol) in DCM (56 mL). The reaction mixture was stirred at r.t. for 2 h, then additional N-hydroxyphthalimide (0.564 g, 3.35 mmol) and EDCI·HCl (0.643 g, 3.35 mmol) were added and the reaction mixture was stirred at r.t. for a further 1.5 h. Water (100 mL) was added, and the layers were separated. The aqueous layer was extracted with DCM (2×25 mL). The organic extracts were combined, passed through a phase separator, and concentrated in vacuo. The crude residue was purified by flash column chromatography, eluting with a gradient of 0-60% EtOAc in hexanes, to give two batches of the title compound (Batch 1: 2.14 g, 46%, ~4:1 ratio of stereoisomers; Batch 2: 1.20 g, 26%, ~25:1 ratio of stereoisomers) as white oily foams. LCMS (Method 1): [M-BOC+H]⁺ m/z 308.2, RT 1.18 minutes.

### Intermediate 5

### tert-Butyl 2-(6-{(S)-(4,4-difluorocyclohexyl)[(2-methylpyrazole-3-carbonyl)aminol-methyl}imidazo[1,2-b][1,2,4]triazin-3-yl)-4-(trideuteriomethoxy)piperidine-1-carboxylate

A solution of Intermediate 4 (~25:1 ratio of stereoisomers) (1.20 g, 2.95 mmol) in DMSO (17.3 mL) was added to a 40 mL screw cap vial under nitrogen containing Intermediate 1 (100 mass %) (650 mg, 1.73 mmol) and {Ir[dF(CF₃)ppy]₂(dtbpy)}PF₆ (38.9 mg, 0.0347 mmol). TFA (0.20 mL, 2.6 mmol) was added next, and the vial cap was quickly replaced. The solution was degassed by bubbling nitrogen through the solution for 10 minutes with a needle out, then the needles were removed and the lid was parafilmed. The reaction mixture was irradiated using a Merck Penn PhD Photoreactor M2 (Settings: Fan=6800 rpm, Stir=800 rpm, LED=100%) at 450 nm for 21.5 h. The reaction mixture was diluted with EtOAc (60 mL) and washed with water (3×60 mL). The combined aqueous layers were extracted with EtOAc (25 mL). The organic layers were combined and washed with brine (60 mL), then passed through a phase separator and concentrated in vacuo. The crude residue was purified by flash column chromatography, eluting with a gradient of 0-100% EtOAc in hexanes, to give the title compound (670 mg, 65%) as an orange oil. LCMS (Method 1): [M+H]⁺ m/z 592.4, RT 1.12 minutes.

### Intermediate 6

### tert-Butyl 2-(6-{(S)-(4,4-difluorocyclohexyl)[(2-methylpyrazole-3-carbonyl)aminol-methyl}imidazo[1,2-b][1,2,4]triazin-3-yl)-4-methoxypiperidine-1-carboxylate

Prepared from Intermediate 1 and O¹-tert-Butyl O²-(1,3-dioxoisoindolin-2-yl) 4-methoxypiperidine-1,2-dicarboxylate in an analogous manner to that described for Intermediate 5.

### Examples 1 & 2

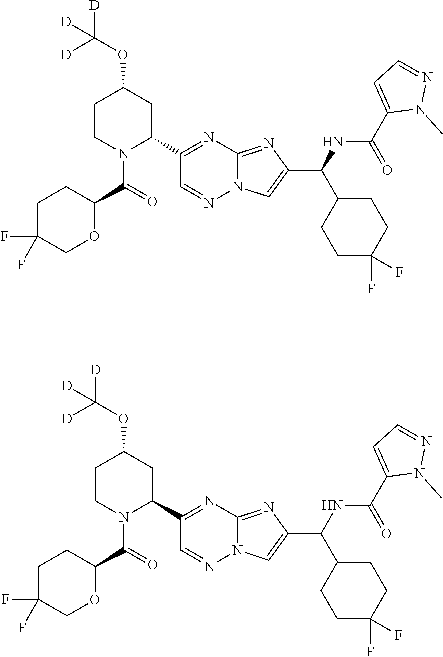 [SMILES: [2H]C([2H])([2H])O[C@H]1CCN(C(=O)[C@@H]2CCC(F)(F)CO2)[C@@H](c2cnn3cc([C@@H](NC(=O)c4ccnn4C)C4CCC(F)(F)CC4)nc3n2)C1 ; [2H]C([2H])([2H])O[C@H]1CCN(C(=O)[C@@H]2CCC(F)(F)CO2)[C@H](c2cnn3cc(C(NC(=O)c4ccnn4C)C4CCC(F)(F)CC4)nc3n2)C1]

### N—[(S)-(4,4-Difluorocyclohexyl)(3-{(2R,4S or 2S,4R)-1-[(2S)-5,5-difluorotetrahydro-pyran-2-carbonyl]-4-(trideuteriomethoxy)piperidin-2-yl}imidazo[1,2-bl[1,2,4]triazin-6-yl)methyl]-2-methylpyrazole-3-carboxamide

### N—[(S)-(4,4-Difluorocyclohexyl)(3-{(2S,4S or 2R,4R)-1-[(2S)-5,5-difluorotetrahydro-pyran-2-carbonyl]-4-(trideuteriomethoxy)piperidin-2-yl}imidazo[1,2-bl[1,2,4]triazin-6-yl)methyl]-2-methylpyrazole-3-carboxamide

Intermediate 5 (670 mg, 1.13 mmol) was dissolved in hydrochloric acid (4M in 1,4-dioxane) (10.3 mL, 41.2 mmol) under nitrogen. The reaction mixture was stirred at r.t. for 10 minutes, then DCM (22.7 mL) was added to aid solubility. After stirring at r.t. for a further 50 minutes, diethyl ether (150 mL) was added. The resulting suspension was stirred vigorously for 5 minutes, then the mixture was filtered under vacuum. The resulting pale yellow solid was washed with diethyl ether (4×20 mL). The resulting pale yellow solid was dissolved in DMF (11.3 mL) and transferred to a flask containing (2S)-5,5-difluorotetrahydropyran-2-carboxylic acid (226 mg, 1.36 mmol). DIPEA (0.98 mL, 5.6 mmol) was added, then HATU (517 mg, 1.36 mmol). The reaction mixture was stirred at r.t. under air for 0.5 h, then diluted with EtOAc (50 mL) and washed with water (3×50 mL). The combined aqueous layers were extracted with EtOAc (30 mL). The organic extracts were combined and washed with brine (30 mL), then passed through a phase separator and concentrated in vacuo. The crude residue was purified by flash column chromatography, eluting with a gradient of 0-100% EtOAc in hexanes, then subjected to chiral SFC (Lux Cellulose-1, 250×21.2 mm, 5 μm, eluting with 3-40% MeOH for a 100 mL/minute, 7.5 minute run time). The fractions containing the separated stereoisomers were concentrated in vacuo and lyophilised from acetonitrile/water (~1:1) to give the title compounds (Peak 1, 61 mg, 8%; Peak 2, 180 mg, 25%) as pale yellow amorphous solids.

Peak 1 (arbitrarily assigned 2R,4S): δ_{H} (400 MHz, DMSO-d₆) (rotamers observed in 0.6:0.4 ratio) 8.83 (s, 0.4H), 8.81 (d, J 6.9 Hz, 0.6H), 8.79 (d, J 6.8 Hz, 0.4H), 8.64 (s, 0.6H), 8.24 (d, J 6.7 Hz, 1H), 7.46 (d, J 2.1 Hz, 1H), 7.06-7.02 (m, 1H), 5.66 (dd, J 6.8, 3.1 Hz, 0.6H), 5.52 (d, J 6.1 Hz, 0.4H), 5.18 (td, J 8.6, 4.7 Hz, 1H), 4.69-4.58 (m, 0.6H), 4.33-4.17 (m, 0.8H), 4.07-3.97 (m, 3.6H), 3.94-3.70 (m, 2H), 3.66-3.54 (m, 1.6H), 3.06-2.88 (m, 0.8H), 2.76-2.65 (m, 0.6H), 2.31-1.48 (m, 14H), 1.46-1.21 (m, 2H). ¹⁹F NMR (282 MHz, DMSO-d₆) δ −90.20 (d, J 232.2 Hz, 1F), −99.40 (d, J 233.4 Hz, 1F), −103.06 to −109.27 (m, 2F). LCMS (Method 3): [M+H]⁺ m/z 640.4, RT 1.74 minutes. TOF-MS (positive m/z): [M+H]⁺ calculated for C₂₉H₃₄D₃N₈O₄F₄: 640.3062; found: 640.3060. Chiral analysis (Method 4): RT 3.64 minutes (94.5%).

Peak 2 (arbitrarily assigned 2S,4S): δ_{H} (400 MHz, DMSO-d₆) (rotamers observed in 0.7:0.3 ratio) 8.82 (d, J 9.0 Hz, 0.3H), 8.80 (d, J 9.0 Hz, 0.7H), 8.56 (s, 1H), 8.29 (s, 0.3H), 8.27 (s, 0.7H), 7.47 (d, J 2.0 Hz, 1H), 7.06-7.04 (m, 1H), 5.95 (d, J 4.5 Hz, 0.7H), 5.65 (d, J 5.6 Hz, 0.3H), 5.22-5.14 (m, 1H), 4.64-4.57 (m, 1H), 4.47 (d, J 13.6 Hz, 0.3H), 4.02 (d, J 0.9 Hz, 3.7H), 3.96-3.79 (m, 1.4H), 3.75-3.62 (m, 0.6H), 3.55 (dq, J 10.5, 5.3 Hz, 0.7H), 3.46 (dq, J 10.9, 5.5 Hz, 0.3H), 3.19-3.09 (m, 0.7H), 2.90-2.76 (m, 1.3H), 2.32-1.50 (m, 13H), 1.47-1.11 (m, 3H). ¹⁹F NMR (282 MHz, DMSO-d₆) δ −90.26 (d, J 232.4 Hz, 1F), −99.31 (d, J 233.2 Hz, 1F), −104.21 to −105.39 (m, 2F). LCMS (Method 3): [M+H]⁺ m/z 640.4, RT 1.72 minutes. TOF-MS (positive m/z): [M+H]⁺ calculated for C₂₉H₃₄D₃N₈O₄F₄: 640.3062; found: 640.3059. Chiral analysis (Method 4): RT 4.22 minutes (99.8%).

### Examples 3 & 4

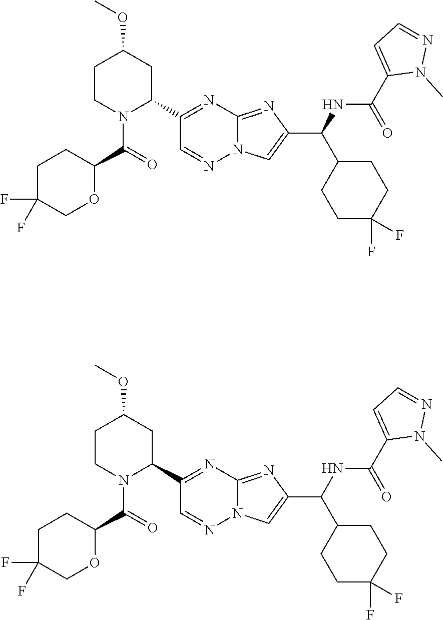 [SMILES: CO[C@H]1CCN(C(=O)[C@@H]2CCC(F)(F)CO2)[C@@H](c2cnn3cc([C@@H](NC(=O)c4ccnn4C)C4CCC(F)(F)CC4)nc3n2)C1 ; CO[C@H]1CCN(C(=O)[C@@H]2CCC(F)(F)CO2)[C@H](c2cnn3cc(C(NC(=O)c4ccnn4C)C4CCC(F)(F)CC4)nc3n2)C1]

### N—[(S)-(4,4-Difluorocyclohexyl)(3-{(2R,4S or 2S,4R)-1-[(2S)-5,5-difluorotetrahydro-pyran-2-carbonyl]-4-methoxypiperidin-2-yl}imidazo[1,2-b][1,2,4]triazin-6-yl)methyl]-2-methylpyrazole-3-carboxamide

### N—[(S)-(4,4-Difluorocyclohexyl)(3-{(2S,4S or 2R,4R)-1-[(2S)-5,5-difluorotetrahydro-pyran-2-carbonyl]-4-methoxypiperidin-2-yl}imidazo[1,2-bl[1,2,4]triazin-6-yl)methyl]-2-methylpyrazole-3-carboxamide

Intermediate 6 (206 mg, 0.350 mmol) was dissolved in hydrochloric acid (4M in 1,4-dioxane) (3.2 mL, 13 mmol) under nitrogen, and DCM (1.8 mL) was added to aid solubility. The reaction mixture was stirred at r.t. for 0.5 h, then diethyl ether (50 mL) was added to precipitate the product fully. The mixture was stirred vigorously for 5 minutes, then filtered under vacuum. The resulting pale yellow solid was washed with diethyl ether (4×5 mL). The pale yellow solid was dissolved in DMF (3.5 mL), and the resulting solution was added to a flask containing (2S)-5,5-difluorotetrahydropyran-2-carboxylic acid (70 mg, 0.42 mmol). DIPEA (0.30 mL, 1.7 mmol) was added, then HATU (160 mg, 0.420 mmol). The reaction mixture was stirred at r.t. for 0.5 h, then diluted with EtOAc (50 mL) and washed with water (3×50 mL). The organic layer was passed through a phase separator and concentrated in vacuo. The crude residue was purified by flash column chromatography, eluting with a gradient of 0-100% EtOAc in hexanes, then by chiral SFC (Lux Cellulose-1, 250×21.2 mm, 5 μm, eluting with 3-40% MeOH for a 100 mL/minute, 7.5 minute run time). The fractions containing the separated stereoisomers were concentrated in vacuo and lyophilised from acetonitrile/water (~1:1) to give the title compounds (Peak 1, 18 mg, 8%; Peak 2, 84 mg, 38%) as pale yellow solids.

Peak 1 (arbitrarily assigned 2R,4S): δ_{H} (400 MHz, DMSO-d₆) (rotamers observed in 0.6:0.4 ratio) 8.83 (s, 0.4H), 8.81 (d, J 7.1 Hz, 0.6H), 8.79 (d, J 7.0 Hz, 0.4H), 8.64 (s, 0.6H), 8.24 (d, J 6.7 Hz, 1H), 7.46 (d, J 2.1 Hz, 1H), 7.06-7.02 (m, 1H), 5.66 (dd, J 6.7, 3.1 Hz, 0.6H), 5.52 (d, J 6.1 Hz, 0.4H), 5.23-5.14 (m, 1H), 4.65 (dd, J 9.0, 3.7 Hz, 0.6H), 4.31-4.19 (m, 0.8H), 4.07-3.97 (m, 3.6H), 3.94-3.70 (m, 2H), 3.66-3.54 (m, 1.6H), 3.05-2.89 (m, 3.8H), 2.76-2.66 (m, 0.6H), 2.31-1.48 (m, 14H), 1.46-1.21 (m, 2H). ¹⁹F NMR (282 MHz, DMSO-d₆) δ −90.22 (d, J 232.2 Hz, 1F), −99.40 (d, J 231.9 Hz, 1F), −102.05 to −108.45 (m, 2F). LCMS (Method 3): [M+H]⁺ m/z 637.4, RT 1.75 minutes. TOF-MS (positive m/z): [M+H]⁺ calculated for C₂₉H₃₇N₈O₄F₄: 637.2874; found: 637.2881. Chiral analysis (Method 4): RT 3.68 minutes (100%).

Peak 2 (arbitrarily assigned 2S,4S): δ_{H} (400 MHz, DMSO-d₆) (rotamers observed in 0.7:0.3 ratio) 8.83 (d, J 8.8 Hz, 0.3H), 8.81 (d, J 9.1 Hz, 0.7H), 8.56 (d, J 1.2 Hz, 1H), 8.29 (s, 0.3H), 8.27 (s, 0.7H), 7.47 (d, J 2.0 Hz, 1H), 7.07-7.03 (m, 1H), 5.98-5.92 (m, 0.7H), 5.65 (d, J 5.7 Hz, 0.3H), 5.22-5.14 (m, 1H), 4.65-4.57 (m, 1H), 4.47 (d, J 13.7 Hz, 0.3H), 4.05-3.97 (m, 3.7H), 3.94-3.79 (m, 1.4H), 3.76-3.63 (m, 0.6H), 3.62-3.52 (m, 0.7H), 3.51-3.42 (m, 0.3H), 3.26 (s, 2.1H), 3.23 (s, 0.9H), 3.19-3.09 (m, 0.7H), 2.90-2.76 (m, 1.3H), 2.32-1.50 (m, 13H), 1.47-1.11 (m, 3H). ¹⁹F NMR (282 MHz, DMSO-d₆) δ −90.26 (d, J 231.9 Hz, 1F), −99.31 (d, J 233.9 Hz, 1F), −102.40 to −107.50 (m, 2F). LCMS (Method 3): [M+H]⁺ m/z 637.4, RT 1.72 minutes. TOF-MS (positive m/z): [M+H]⁺ calculated for C₂₉H₃₇N₈O₄F₄: 637.2874; found: 637.2878. Chiral analysis (Method 4): RT 4.28 minutes (99.4%).

1. A compound of formula (I), or a pharmaceutically acceptable salt thereof: 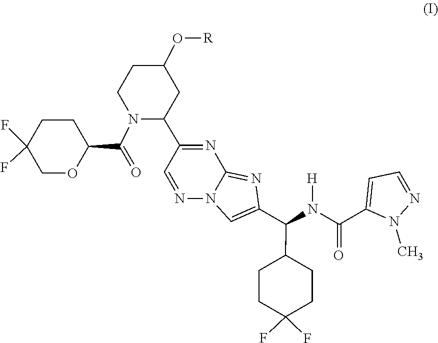
wherein R represents methyl.

2. A compound of formula (I) as defined in claim 1, or a pharmaceutically acceptable salt thereof, wherein R represents CH₃.

3. A compound of formula (I) as defined in claim 1, or a pharmaceutically acceptable salt thereof, wherein R represents CD₃.

4. (canceled)

5. (canceled)

6. (canceled)

7. A pharmaceutical composition comprising a compound of formula (I) as defined in claim 1, or a pharmaceutically acceptable salt thereof, in association with a pharmaceutically acceptable carrier.

8. (canceled)

9. (canceled)

10. A method for the treatment and/or prevention of disorders for which the administration of a modulator of IL-17 function is indicated, which comprises administering to a patient in need of such treatment an effective amount of a compound of formula (I) as defined in claim 1, or a pharmaceutically acceptable salt thereof.

11. A method for the treatment and/or prevention of an inflammatory or autoimmune disorder, which comprises administering to a patient in need of such treatment an effective amount of a compound of formula (I) as defined in claim 1, or a pharmaceutically acceptable salt thereof.
